(19) United States
(12) Patent Application Publication
(10) Pub. No.: US 2019/0390403 A1
(43) Pub. Date: Dec. 26, 2019

(54) WIRE ROPE WITH ENHANCED WIRE WRAP

(71) Applicant: Intuitive Surgical Operations, Inc., Sunnyvale, CA (US)

(72) Inventor: Andrew C. WATERBURY, Sunnyvale, CA (US)

(21) Appl. No.: 16/481,415

(22) PCT Filed: Jan. 26, 2018
(86) PCT No.: PCT/US2018/015474

§ 371 Date: Jul. 26, 2019

Related U.S. Application Data
(60) Provisional application No. 62/451,039, filed on Jan. 26, 2017.

(51) Int. Cl. D07B 1/06; A61B 34/00; A61B 34/35

(52) U.S. Cl. CPC D07B 1/068; A61B 34/71; A61B 34/35; A61B 2034/305; D07B 2201/1036; D07B 2201/2061; D07B 2201/1014

(57) ABSTRACT

A wire rope comprising: a core that includes a plurality wires; and at least thirteen outer strands that each includes a plurality of wires.

### CLAIM OF PRIORITY

This application claims the benefit of priority to U.S. Provisional Patent Application No. 62/451,039, filed on Jan. 26, 2017, which is hereby incorporated by reference herein in its entirety.

### BACKGROUND

Wire rope is a complex intricate machine. Wire ropes generally include three components: a wire, wire strand and core. A wire can be formed from a metal such as stainless steel or tungsten, for example. A wire strand is generally formed by helically winding several wires around a central wire. Several outer strands, in turn, are helically wound about a core to form the complete wire rope structure. As disclosed in U.S. Pat. No. 3,092,956, entitled, “7-Strand Wire Rope”, a core may be fibrous or may include an inner wire strand. Typical wire rope constructions include six outer strands, eight outer strands or twelve outer strands.

FIG. 1 is an illustrative perspective view of an example wire rope 100 shown partially unwound that includes multiple stranded wires 102 helically wound about a strand core 103 and that includes multiple strands 104 helically wound about a rope core 106. The wire rope includes multiple strands. A stranded wire 102 is shown partially unwound from a strand core wire 103, and a strand 104 is shown partially unwound from the rope core 106. The partially unwound strand 104 includes multiple outer wires 102 helically wound about the strand core wire 103. The wire rope 100 includes multiple strands 104 wound about the core 106. In response to changing stress as the rope 100 is pulled axially and flexed during operation, the helically wound wires 102 within the strands 104 move slightly relative to one another. The strands 104 themselves also slide relative to each other to equalize the more significant stresses within the rope 100. The rope core 106 maintains rope geometry and supports the strands 104 as the wire 102 and strand 104 motions take place, preventing them from collapsing or slipping out of position relative to one another when subjected to radial pressure. As a wire rope 100 is loaded, the helical lay of the strands 104 causes them to press inward toward the rope axis. The core 106 supports this pressure and prevents the strands 104 from rubbing and crushing. The core 106 also maintains the position of the strands 104 during bending.

FIG. 2 is an illustrative perspective view of an example ingle-layer wire strand 104 of the wire rope 100 of FIG. 1. The wire strand 104 includes a multiple wire outer layer that includes six helically would outer wires 102 laid about a central core wire 103 in a radially symmetrical pattern. The example strand 104 includes seven wires of essentially equal diameter packed closely together with six stranded wires 102 laid about the seventh core wire 103. The center core wire 103 may be exactly the same size as the outer wires but is often is slightly larger.

FIG. 3 is an illustrative cross-section view of a first example six outer strand wire rope 300 having six outer strands 304. The first example 300 wire rope has a 7×37 construction. That is, it has seven strands 304, 306, each having thirty-seven wires 302. The first example wire rope 300 includes seven strands: six 1×37 outer strands 302 that are, in turn, stranded about a 1×37 center core strand 304. All wires have the same diameter. The example six outer strand wire rope 300 has an outer diameter (OD) equal to twenty-one wire diameters.

FIG. 4 is an illustrative cross-section view of a second example eight outer strand wire rope 400 having eight outer strands 404. The second example wire rope 400 has an 8×19-7×7 construction. That is, it has eight 1×19 outer strands 404 each having nineteen wires 402 and a core 406 that includes seven 1×7 strands 408, 410, each having seven wires 402. The eight outer strands 404 are stranded about the core 406. The core 406 has seven 1×7 strands: six 1×7 outer strands 408 that are, in turn, stranded together about a center 1×7 strand 410. The example second wire rope 400 has an outer diameter (OD) equal to nineteen wire diameters.

FIG. 5 is an illustrative cross-section view of a third example twelve outer strand wire rope 500 having twelve outer strands 504. The third example wire rope 500 has a 19×19 construction. That is, it has nineteen strands 504, 508, 510, each having nineteen wires 502. The third example wire rope 500 includes twelve 1×19 outer strands 504, each having nineteen wires 502. The third example wire rope 500 has a core 506 that includes seven 1×19 strands. More specifically, the core 506 includes six 11×19 outer strands 508, which in turn, are stranded together and wound about a center 1×19 strand 510. The example third wire rope 500 has an outer diameter (OD) equal to twenty-five wire diameters.

FIG. 6 is an illustrative cross section view of fourth example six outer strand wire rope 600 having six outer strands 604. The fourth example wire rope has a 7×7×7 construction. That is, it has six 7×7 outer strands 604, each having forty-nine wires 602, stranded together about a seventh, center 7×7 core strand 606, having forty-nine wires 602. Each 7×7 strand includes six outer wires 602 stranded about a center core wire 603. Thus, the fourth example 7×7×7 wire rope 600 is constructed by stranding together six 1×7 strands 604 about a core 1×7 strand 606, and each 1×7 strand is constructed by stranding six wires 602 about a core wire 603.

FIG. 7 is an illustrative cross section view of a fifth example six outer wire rope 700 having six outer strands 704. The fifth example wire rope has a 7×7×19 construction. That is, it has six 7×19 outer wire rope strands 704, each having one hundred and thirty-three wires 702, stranded about a 7×19 center wire rope strand 706, having one hundred and thirty-three wires 702. Each 7×19 strand includes six 1×19 outer strands stranded about a 1×19 center core 603. Thus, the fifth example 7×7×19 wire rope 700 is constructed by stranding together six 7×19 strands 704 about a core 7×19 strand 706, and each 7×19 strand is constructed by stranding six 1×19 wire strands about a core 1×19 wire core.

FIG. 8 is an illustrative cross section view of a sixth example six outer wire rope 800 having six outer strands 804. The sixth example wire rope has a 7×(7×19-1×37) construction. That is, it has six 7×19-1×37 outer strands 804, each having one hundred and seventy-one wires 802, stranded together about a center 7×19−1×37 core strand 806 having one hundred and seventy-one wires 802. Each outer strand 804 includes seven 1×19 strands 808 stranded about a 1×37 strand core 810. The core strand 806 also includes seven 1×19 strands 808 stranded about a 1×37 strand core 810. Thus, the sixth example 7×(7×19-1×37) wire rope 800 is constructed by stranding together six 7×19-1×37 outer strands 804 about a 7×19-1×37 core strand 806, and each 7×19-1×37 strand is constructed by stranding seven 1×19 wire strands about a 1×37 wire core.

FIG. 9 is an illustrative cross section view of a seventh example six outer wire rope 900 having six outer strands 904. The seventh example wire rope has a 7×7×37 construction. That is, it has six 7×37 outer strands 904, each having two hundred fifty-nine wires 902, stranded together about a 1×37 center core strand 906 having two hundred fifty-nine wires 902. Each outer strand 904 includes six 1×37 strands 908 stranded about a 1×37 strand core 910. The core strand 906 also includes six 1×37 strands 908 stranded about a 1×37 strand core 810. Thus, the seventh example 7×7×37 wire rope 900 is constructed by stranding together six 7×37 outer strands 904 about a core 7×37 core strand 906, and each 7×37 strand is constructed by stranding six 1×37 wire strands about a 1×37 wire core.

Referring to FIGS. 6-9, it can be seen that the example six outer strand wire ropes 600-900 follow a pattern of stranding together multiple smaller diameter wire ropes into a larger diameter wire rope having a stranding pattern similar to the stranding pattern of the smaller diameter wire ropes. In general, the greater the amount of metal within a wire rope cross section, the greater the tensile strength of the wire rope and the greater its resistance to tensile fatigue. Wire ropes often are formed by stranding together multiple smaller diameter wire ropes, as shown in FIGS. 6-9, into a larger diameter wire rope to increase overall wire rope tensile strength by increasing the total amount of metal while keeping the filament (wire) diameter as small as practically possible.

Still referring to FIGS. 6-9, each outer strand is itself a wire rope stranded about a wire rope core. Moreover, each successive example six outer strand wire ropes 600-900 has a larger total number of wires than the previous example. The fourth example six outer strand wire rope 600 has three hundred and forty-three (343) wires. The fifth example six outer strand wire rope 700 has nine hundred and thirty-one (931) wires. The sixth example six outer strand wire rope 800 has one thousand one hundred and ninety-seven (1,197) wires. The seventh example six outer strand wire rope 900 has one thousand eight hundred and thirteen (1,813) wires. Moreover, the outer strands of each successive example six outer strand wire ropes 600-900 has a larger total number of wires than the previous example. In the fourth example six outer strand wire rope 600, each outer strand 604 has forty-nine wires. In the fifth example six outer strand wire rope 700, each outer strand 704 has one hundred and thirty-three wires. In the sixth example six outer strand wire rope 800, each outer strand 804 has one hundred and seventy-one wires. In the seventh example six outer strand wire rope 900, each outer strand 904 has two hundred fifty-nine wires. Thus, for example, assuming identical wire diameters, the example six outer strand wire ropes 600-900 have successively increased tensile strength and successively increased resistance to tensile fatigue while keeping the filament (wire) diameter as small as practically possible.

Wire size often is selected to achieve tight packing of wires and to achieve stabilization of wire strands. An outer layer wire strand might not fit smoothly onto an inner layer wire strand unless the lay angle of the two layers is slightly different. To simplify the manufacture of multilayered strands, the size of the wires in in each layer is sometimes varied. There are two commonly used stranding techniques of this type. In one method, the number of wires in the outer and inner layers is kept equal so that the outer wires can rest in the valleys of the layer beneath. Thus, the diameter of the second-layer wires is larger than that of the first; the diameter of the third-layer wires, larger than that of the second, and so on. The wires in any one layer, however, are all of the same diameter. This strand type is commonly referred to as a “Seale” wire construction. One limitation for multilayer strands of Scale construction is that the wires hi the outer layers may become so large that the flexibility of the rope is impaired. This problem can be reduced for many single-operation strands if the number of wires in each succeeding layer are doubled to form what is known as a “Warrington” wire construction. If this is done, however, it is often necessary to use two sizes of wire in the outside layer, placing smaller wires on the crowns of the interior wire layer and larger wires in the valleys.

### BRIEF DESCRIPTION OF THE DRAWINGS

FIG 1 is an illustrative perspective view of an example wire rope in which wire strands and are shown partially unwound.

FIG. 2 is an illustrative perspective view of an example single-layer wire strand of the wire rope of FIG. 1.

FIG. 3 is an illustrative cross-section view of a first example six outer strand wire rope,

FIG. 4 is an illustrative cross-section view of a second example eight outer strand wire rope.

FIG. 5 is an illustrative cross-section view of a third example twelve outer strand wire rope.

FIG. 6 is an illustrative cross section view of fourth example six outer strand wire rope.

FIG. 7 is an illustrative cross section view of a fifth example six outer wire strand wire rope.

FIG. 8 is an illustrative cross section view of a sixth example six outer wire rope having six outer strands.

FIG. 9 is an illustrative cross section view of a seventh example six outer wire rope.

FIG. 10 is an illustrative plan view of a minimally invasive teleoperated surgical system.

FIG. 11 is a perspective view of the surgeon's console.

FIGS. 12A-12B are illustrative perspective, partially cut away, views of a pivotable wrist portion of a surgical instrument that mounts an articulable jaw end effector, shown in two different positions.

FIG. 13 is an illustrative drawing representing a wire cable configured to follow a guide surface provided by a pulley and showing tensile and bending stresses acting upon the wire rope.

FIG. 14 is an illustrative cross section view of a thirteen outer strand wire.

FIG. 15 is an illustrative cross section view of a sixteen outer strand wire rope.

FIG. 16 is an illustrative cross section view of a nineteen outer strand wire rope.

FIG. 17 is an illustrative cross section view of a twenty-four outer strand wire rope.

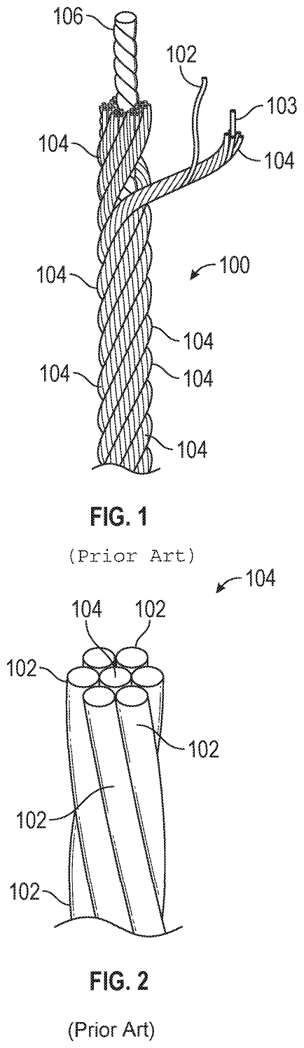

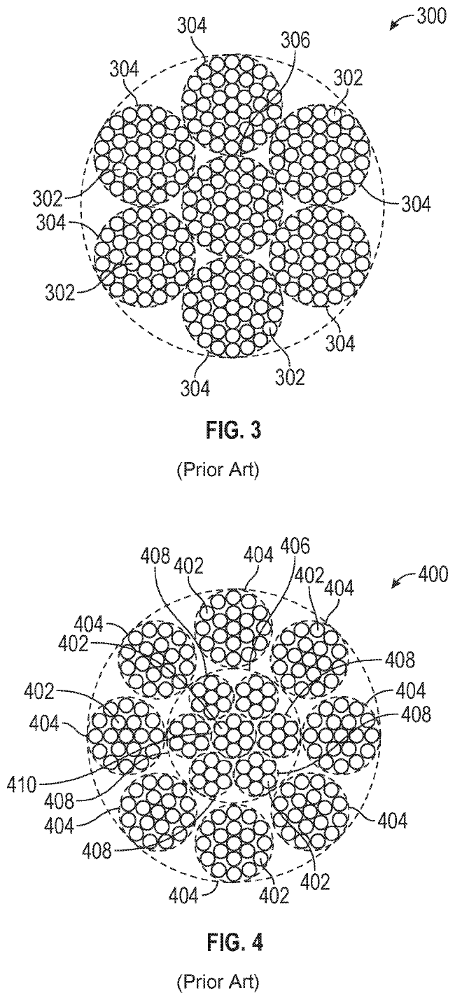

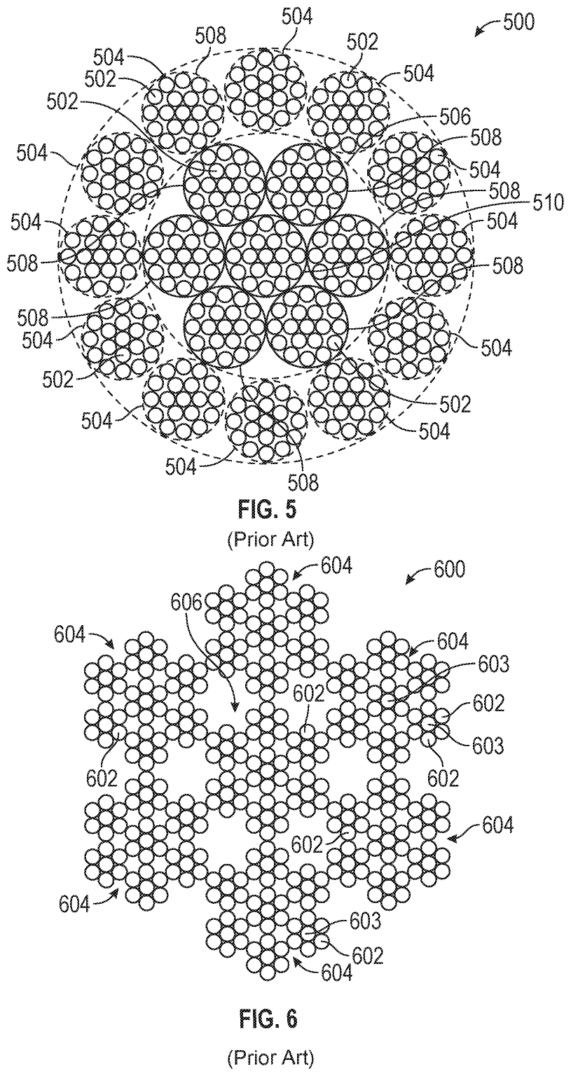

### DESCRIPTION OF EMBODIMENTS

The inventor unexpectedly and surprisingly found that one can significantly increase the tensile strength of a wire rope while minimizing bending stress upon individual wires by increasing the number of outer strands and constructing the wire rope strands with the smallest practical wire size. For wire rope characterized by having large diameter ratios, that is the ratio of the wire rope diameter to the diameter of the wire used to construct the wire rope, the tensile strength is increased due to increased wire packing factor while bending stress is minimized due to small diameter of the individual wires that make up the wire rope. As used herein, a “wire packing factor” refers to a fraction of a total cross-section area of a wire rope that is filled with wire material, typically metal.

Surgical instruments used in teleoperated minimally invasive surgery (MIS) often mimic the motions of the human hand. Teleoperation refers to operation of a machine at a distance in which an endoscope that includes a camera to provide a view of a surgical site within a patient's body. Kinematic transformations are used to translate full-scale hand motions of a surgeon to corresponding small-scale motions of a tiny surgical instrument operative at a surgical site within a human body cavity. Movement distances of a surgeon's hands may be scaled by a factor of about 1:3, for example, to translate those large-scale hand movement distances to corresponding small-scale surgical instrument movement distances. Mechanical mechanisms to create motions that mimic large-scale human hand movements with small-scale surgical instrument movements have inherently have small features. Small-scale surgical instrument motions typically are driven by wire ropes, sometimes referred to in the MIS realm as tendons or cables, that are tolerant to the small bend radii on the order of an instrument radius or smaller while still being able to transfer the relatively larger forces required for activities such as cutting, stapling or suturing, for example.

The small surgical instrument dimensions required for operation within an MIS environment require use of small-diameter wire rope. These wire rope diameter limitations together with usage constrains including tensile stress, sensitivity to bending stress and wear resistance motivated the inventor to explore alternative wire rope configurations of small-diameter wire. Manufacturing limitations impose practical limitations upon the minimum diameter of wires within a wire rope. Minimum wire diameter is generally material-dependent. Production yield or cost may make it impractical to use the smallest possible diameter wire for a given wire material. Better tensile strength, while decreasing bending stress, generally may be achieved by positioning smaller diameter wires contained within a wire rope near the outer periphery of the wire rope. In general, the greater the amount of wire material within a wire rope cross-section, the greater will be the tensile strength of the wire rope.

The inventor observed that in general, a wire rope construction for an MIS surgical instrument should provide a high enough tensile strength to enable the exertion of clinically relevant forces while maintaining high mechanical fatigue life. In particular, the wire rope construction generally should have as large a diameter as instrument dimensions and wrist mechanism dimensions will allow to maximize tensile strength. Furthermore, the inventor observed that a wire rope construction for an MIS surgical instrument generally should minimize sensitivity to bending to achieve high fatigue life. Specifically, wires that make up a wire rope construction should be as small as practically possible to minimize bending stresses. Decreased wire diameter generally results in reduced bending stress, increasing fatigue life. Moreover, the inventor observed that a wire rope construction for an MIS surgical instrument should provide a large enough surface area to minimize external wear against controlling surfaces and to minimize internal wear from wires that make up the wire rope sliding against one another. In general, this means having as many outer strands as is practically possible.

The inventor discovered the unexpected result that for small-diameter wires within small-diameter wire ropes used in MIS surgical instruments, for example, for a given wire rope diameter and a given smallest wire-diameter within the wire rope, increased wire rope tensile strength and reduced bending stress are better achieved through increasing the number of outer strands of a wire rope having the given smallest wire-diameter than through stranding together multiple smallest-diameter wire ropes into a wire rope having the given wire rope diameter. More specifically, the inventor explored a variety of different wire rope configurations and discovered the unexpected result that a significant increase in tensile strength with minimal sensitivity of the wire rope to bending stress fatigue may be achieved, for a wire rope having a given diameter, by providing in the wire rope at least thirteen outer strands having a single wire-diameter in which an overall-rope-diameter-to-outer-strand-wire-diameter ratio, which represents a ratio of overall wire rope diameter to individual outer strand wire-diameter is at least twenty-seven. Stated differently, the ratio represents the number of outer strand wire-diameters aligned side-by-side to span the overall wire rope diameter, which is at least twenty-seven.

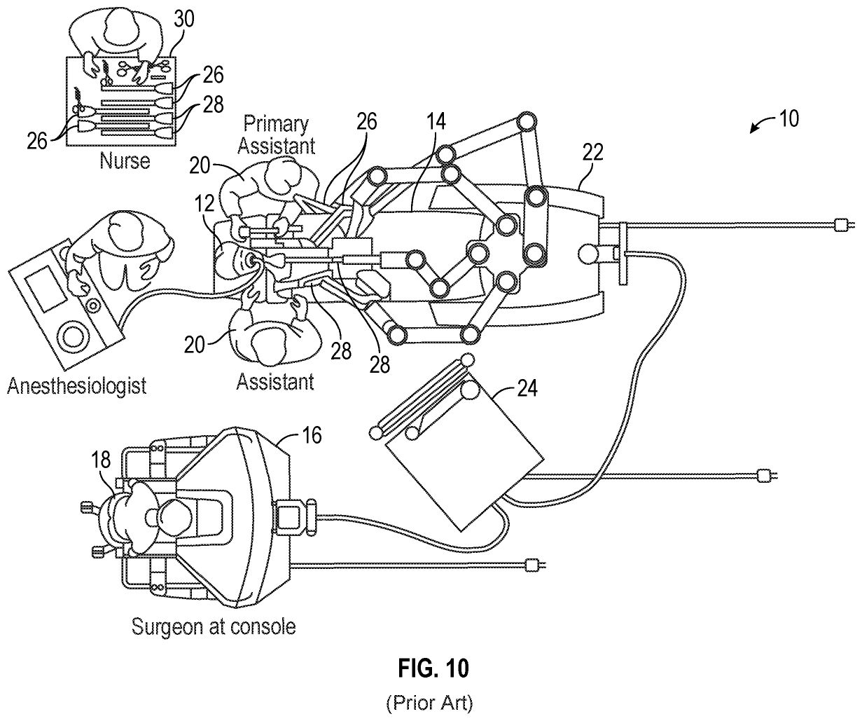

FIG. 10 is an illustrative plan view of a minimally invasive teleoperated surgical system 10, typically used for performing a minimally invasive diagnostic or surgical procedure on a patient 12 who is lying on an operating table 14. The system includes a surgeon's console 16 for use by a surgeon 18 during the procedure. One or more assistants 20 may also participate in the procedure. The minimally invasive teleoperated surgical system 10 further includes a patient-side cart(s) 22 and an electronics cart 24. The patient-side cart 22 can manipulate at least one surgical instrument 26 through a minimally invasive incision in the body of the patient 12 while the surgeon 18 views the surgical site through the surgeon's console 16. An image of the surgical site can be obtained by an endoscope 28, such as a stereoscopic endoscope, which can be manipulated by the patient-side cart 22 to orient the endoscope 28. Computer processors located on the electronics cart 24 can be used to process the images of the surgical site for subsequent display to the surgeon 18 through the surgeon's console 16. In some embodiments, stereoscopic images can be captured, which allow the perception of depth during a surgical procedure. The number of surgical instruments 26 used at one time will generally depend on the diagnostic or surgical procedure and the space constraints within the operative site among other factors. If it is necessary to change one or more of the surgical instruments 26 being used during a procedure, an assistant 20 can remove the surgical instrument 26 from the patient-side cart 22, and replace it with another surgical instrument 26 from a tray 30 in the operating room.

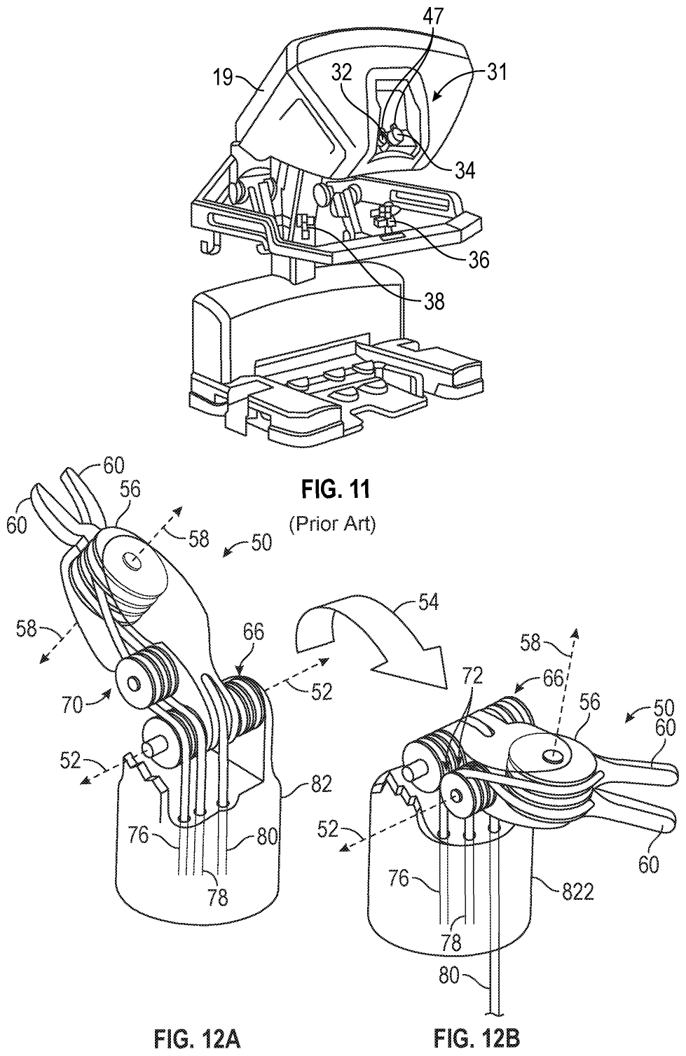

FIG. 11 is a perspective view of the surgeon's console 16. The surgeon's console 16 includes a viewer display 31 that includes a left eye display 32 and a right eye display 34 for presenting the surgeon 18 with a coordinated stereoscopic view of the surgical site that enables depth perception. The console 16 further includes one or more hand-operated control inputs 36 to receive the larger-scale hand control movements. One or more surgical instruments installed for use on the patient-side cart 22 move in smaller-scale distances in response to surgeon 18's larger-scale manipulation of the one or more control inputs 36. The control inputs 36 can provide the same mechanical degrees of freedom as their associated surgical instruments 26 to provide the surgeon 18 with telepresence, or the perception that the control inputs 36 are integral with the instruments 26 so that the surgeon has a strong sense of directly controlling the instruments 26. To this end, position, force, and tactile feedback sensors (not shown) may be employed to transmit position, force, and tactile sensations from the surgical instruments 26 back to the surgeon's hands through the control inputs 36, subject to communication delay constraints.

FIGS. 12A-12B are illustrative perspective, partially cut away, views of a pivotable wrist portion 50 of a surgical instrument that mounts an articulable jaw end effector, shown in two different positions. The surgical instrument includes a shaft on which the wrist portion is mounted. The wrist portion includes a first pulley set 70, a second pulley set 66, 72, and a third pulley 74 set to guide first, second and third wire rope segments 76, 78, 80 that extend from within a shaft 82 and about the pulley sets. The wire ropes 76, 78, 80 are used in combination to cause the wrist portion 50 to pivot about a first axis 52 as indicated by the arrow 54, for example. The wire ropes 76, 78, 80 also are used in combination to cause the end effector portion 56 of the wrist portion 50 to pivot about a second axis 58. The end effector 56 includes jaws 60. It will be appreciated that tensile forces are imparted to the wire ropes 76, 78, 80 as they are used to pull the wrist 50 between pivot positions and as they are used to pivot the end effector 56. Moreover, it will be appreciated that the wire ropes 76, 78, 80 follow a tortuous (i.e. circuitous with sharp curves) paths over several different sets of pulley guide surfaces and that movement of the wire ropes 76, 78, 80 along those paths imparts bending stresses to the wire ropes. Details of an embodiment of the wrist portion 50 of the surgical instrument are provided in U.S. Pat. No. 6,394,998, entitled, “Surgical Tools for Use in Minimally Invasive Telesurgical Applications”.

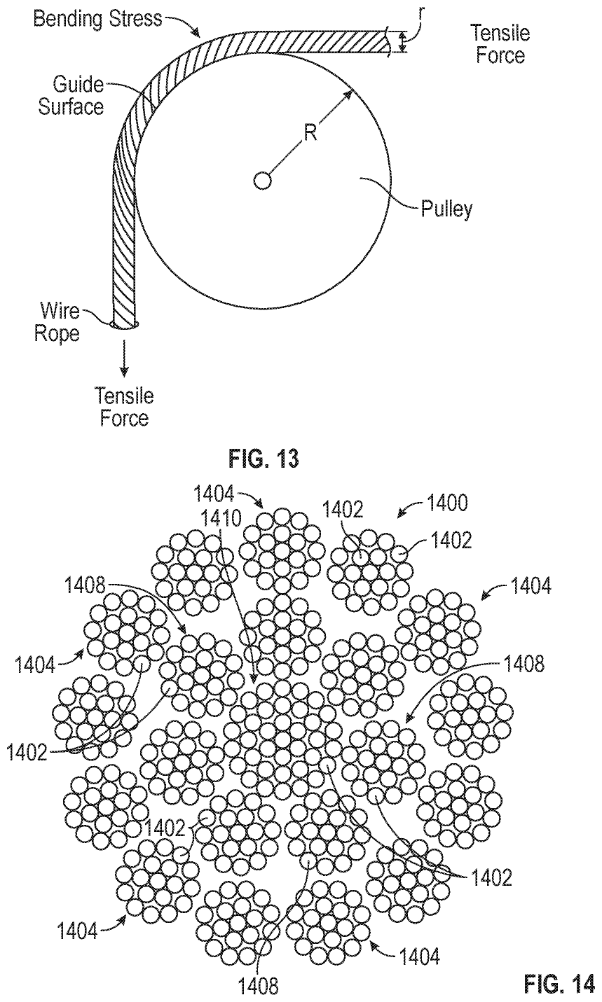

FIG. 13 is an illustrative drawing representing a single wire configured to follow a guide surface provided by a pulley and showing tensile and bending stresses acting upon the wire. Bending stress can be represented by the following expression.

σ_{b≈E*r/R} (1)

where σ_{b} represents bending stress imparted to a wire, r represents radius of individual wires, R is the radius of the pulley, and E represents young's modulus. It will be appreciated that the larger the radius of the wire, the larger the bending stress. Thus, use of smaller wires reduces wire fatigue due to bending stress. It will be further appreciated, however, that the smaller the diameter of individual wire, the less tensile strength it possesses, and therefore, a large number of smaller wires is required to provide minimal sensitivity to bending fatigue while also providing sufficient tensile strength. A MIS surgical instrument in accordance with some embodiments has a shaft diameter in the range 4-10 mm. The MIS surgical instrument includes a wire rope that includes an inner core that includes a plurality of core wires and that has a diameter in a range 0.241-1.697 mm. The wire rope has an outer wrap including at least thirteen outer strands, each including a plurality of outer strand wires and each outer strand having a diameter in the range 0.046-0.229 mm. The MIS surgical instrument has an end effector having a bend portion, such as the example wrist 50 that is rotatable about the first axis 52 and the jaws that are rotatable about the second axis 58, having a maximum bending radius equal to half the diameter of the instrument shaft 82. In an MIS surgical instrument in accordance with some embodiments, a wire rope bends through an angle of at least fifteen degrees. An actuator such as a motor (not shown) imparts a tensile force in the range 44-445 N upon the wire rope to impart a force with a strain smaller than 0.02.

Table A sets forth wire rope dimensions suitable for surgical instruments having a range of 13-24 outer strand wire ropes and shaft diameters in a range 4-10 in accordance with some embodiments. The number of outer strand wire-diameters to span the overall wire rope diameter for the wire ropes in Table A range from about 27-81. The ranges for the dimensional values in Table A is due to the various possible outer strand configurations as well as the range of wire diameters associated with appropriate wire rope materials for medical use. Some materials can be drawn into finer wire than others.

TABLE A

| | | | | E |
|---|---|---|---|---|
| A | B | C | D | inst. shaft |
| # of outer | core diam. | outer strand | overall cable | diam. |
| strands | [mm] | diam. [mm] | diam. [mm] | [mm] |
| 13 | .241-.823 | .076-.229 | .394-1.281 | 4-10 |
| 14 | .267-.902 | .076-.229 | .419-1.378 | 4-10 |
| 15 | .290-.980 | .076-.229 | .442-1.438 | 4-10 |
| 16 | .315-1.059 | .076-.229 | .467-1.517 | 4-10 |
| 17 | .284-1.140 | .064-.229 | .411-1.598 | 4-10 |
| 18 | .305-1.219 | .064-.229 | .432-1.677 | 4-10 |
| 19 | .325-1.298 | .064-.229 | .452-1.756 | 4-10 |
| 20 | .345-1.379 | .064-.229 | .472-1.837 | 4-10 |
| 21 | .366-1.458 | .064-.229 | .493-1.916 | 6-10 |
| 22 | .386-1.539 | .064-.229 | .513-1.997 | 6-10 |
| 23 | .406-1.618 | .064-.229 | .533-2.076 | 6-10 |
| 24 | .305-1.697 | .046-.229 | .407-2.155 | 6-10 |

Table B sets forth core diameter manges and outer strand diameter manges for surgical instrument shaft diameters in the range 4-10 mm for some wire rope embodiments with thirteen, sixteen, nineteen and twenty-four outer strands counts in accordance with some embodiments. The range of values in Table B are because of the range of wire diameters associated with appropriate wire rope materials for medical instruments. For the wire ropes in Table B, the number of outer strand wire-diameters to span the overall wire rope diameter range from 27-63. That is, the number of outer strand wires lined up side-by-side to span the overall wire rope diameter is in the range 27-63. In some embodiments, internal wires may have a different wire-diameter than the wire-diameter of the outer strand wires.

TABLE B

| | B | C | D |
|---|---|---|---|
| A | Cable diam. | Outer strand | Core diam. |
| Construction | [mm] | diam. [mm] | [mm] |
| 13 × 19-7 × 19-1 × 37 | .411-.686 | .076-.127 | .259-.432 |
| 16 × 37-16 × 19-7 × 37 | .686-1.143 | .107-.178 | .472-.787 |
| 19 × 37-20 × 19-13 × 19-7 × | .778-1.296 | .107-.178 | .564-.940 |
| 19-1 × 37 | | | |
| 24 × 37-18 × 37-12 × 37-7 × | .961-1.601 | .107-.178 | .747-1.245 |
| 37 | | | |

FIG. 14 is an illustrative cross section view of a thirteen outer strand wire rope 1400. The thirteen outer strand wire rope 1400 has wires arranged in a 13×19−7×19−1×37 construction. The thirteen outer strand wire rope 1400 has an outer strand construction, also referred to herein as a ‘wrap’ about a core. The wrap that includes thirteen outer strands 1404, each having nineteen wires 1402. The thirteen outer strand wire rope 1400 has a core region that includes first inner layer of strands 1408 that each includes nineteen wires 1402. The thirteen outer strand wire rope 1400 core region includes an inner core 1410 that has thirty-seven wires 1402. The thirteen outer strand wire rope 1400 includes a total of four hundred and seventeen (417) wires. The thirteen outer strand wire rope 1400 has a wire diameter of approximately 0.015-0.025 mm.

In some embodiments, the diameter of wires in the outer strands 1404 is equal to the diameter of wires in the inner layer strands 1408 and the wires in the core 1410. In other embodiments, the diameter of wires in the outer strands 1404 is less than the diameter of wires in one or both of the inner layer strands 1408 and wires in the core 1410. In some embodiments, wires in one or both of the inner layer strands 1408 and wires in the core 1410 are in a range 1.08 and 1.12 times the diameter of wires in the outer strands 1404.

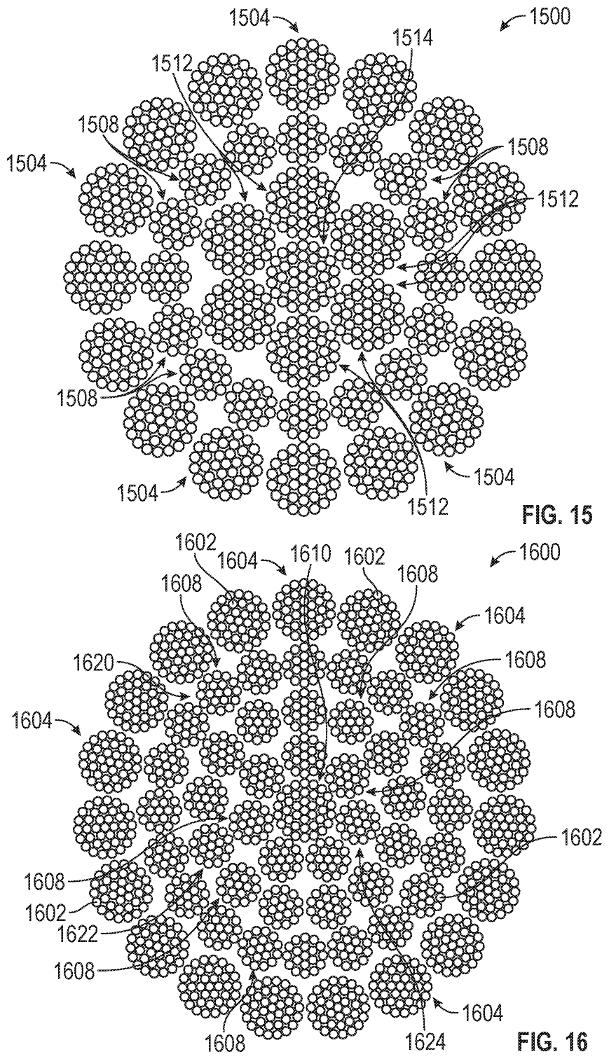

FIG. 15 is an illustrative cross section view of a sixteen outer strand wire rope 1500. The sixteen outer strand wire rope has wires arranged in a 16×37−16×19-7×37 construction. The sixteen outer strand wire rope 1500 has an outer strand construction, or wrap. The wrap includes sixteen outer strands 1504, each having thirty-seven wires 1502. The sixteen outer strand wire rope 1500 includes a core region that has a first inner layer of strands 1508 that each includes nineteen wires 1502. The thirteen outer strand wire rope 1500 core region includes has a 7×37 inner core layer that includes six 1×37 strands 1512 wrapped about a 1×37 core 1514. The sixteen outer strand wire rope 1500 includes a total of one thousand one hundred and fifty-two (1,152) wires. The sixteen outer strand wire rope 1500 has a wire diameter of approximately 0.015-0.025 mm.

FIG. 16 is an illustrative cross section view of a nineteen outer strand wire rope 1600. The nineteen outer strand wire rope 1600 has wires arranged in a 19×37-20×19-13×19-7×19-1×37 construction. The nineteen outer strand wire rope 1600 has a 1×37 outer strand construction (wrap), which includes nineteen outer strands 1604, each having thirty-seven wires 1602. The nineteen outer strand wire rope 1600 has a core region that has three successive inner layers of 1×19 strands. A first inner layer 1620 has twenty (20) 1×19 strands 1608, each having nineteen wires 1602. A second inner layer 1622 has thirteen (13) 1×19 strands 1608, each having nineteen wires 1602. A third inner layer 1624 has seven (7) 1×19 strands 1608, each having nineteen wires 1602. The nineteen outer strand wire rope 1600 has an inner core 1310 having thirty-seven (37) wires 1602. The nineteen outer strand wire rope 1600 includes a total of one thousand three hundred and sixty-seven (1,367) wires 1602. The nineteen outer strand wire rope 1600 has a wire diameter of approximately 0.015-0.025 mm.

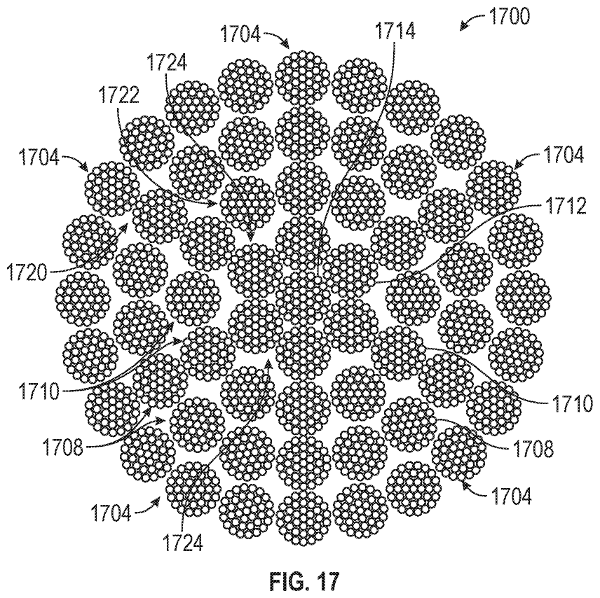

FIG. 17 is an illustrative cross section view of a twenty-four outer strand wire rope 1700. The twenty-four outer strand wire rope 1700 has wires arranged in a 24×37-18×37-12×37-7×37 construction. The twenty-four outer strand wire rope 1700 has a 24×37 outer strand construction (wrap), which includes twenty-four outer strands 1704, each having thirty-seven wires 1702 The twenty-four outer strand wire rope 1700 has a core region with two successive inner layers of 1×37 strands. A first inner layer 1720 has eighteen (18) 1×37 strands 1708, each having thirty-seven wires 1702. A second inner layer 1722 has twelve (12) 1×37 strands 1710, each having thirty-seven wires 1702. A 7×37 inner core 1724 has six 1×37 strands 1712 wrapped about a 1×37 inner core strand 1714. The twenty-four outer strand wire rope 1700 includes a total of two-thousand, two hundred and fifty-seven (2,257) wires 1602. The twenty-four outer strand wire rope 1700 has a wire diameter of approximately 0.015-0.025 mm.

The wires of the wire rope embodiments of FIGS. 14-17 are metal. Robotic medical instruments require metals used in wire rope to be biocompatible and corrosion resistant. The metals must also have high tensile strengths, be resistant to wear, have a reasonably high Elastic modulus, and the ability to be drawn down to ultra-fine wire sizes. Example metals of suitable metals include titanium alloys, stainless steel alloys, tungsten alloys, and super alloys such as Haynes 25 and MP35N,

Table C compares wire rope constructions that have common diameters as indicated by columns B and E for a given row. The wire rope constructions in columns A and D are made using the same wire diameter, so the constructions in each row of Table C have the same ratio of the wire rope diameter to the outer strand wire diameter (D/d), which can also be described as the number of outer strand wire-diameters to span the overall wire rope diameter, as seen in columns C and G. All the calculated diameters and strengths in Table C assume the wire ropes are made from 0.0254 mm diameter 304 stainless steel wire with a 2.62 GP (i.e. gigapascal which is a pressure equivalent to 1 e 9 N/m²) ultimate tensile strength.

Table C shows that for a given wire rope (cable) diameter with a small relative wire diameter such that stranding smaller wire rope into larger diameter wire rope becomes practical, a larger number of wires can be packed into a wire rope by adding increasingly more outer strands than is achieved from stranding together smaller diameter wire ropes into a larger diameter wire rope that has a stranding pattern similar to the smaller diameter wire ropes. Table C also shows that a cable with a larger number of outer stands, for a given wire rope diameter and a given wire diameter, has greater tensile strength than a wire rope with the same wire rope diameter and the same wire diameter that is produced by stranding together smaller diameter wire ropes into a larger diameter wire rope that has a stranding pattern similar to the smaller diameter wire ropes. In accordance with some embodiments, core wires have a diameter in a range of about 1.0 times as large to 1.12 times as large as a diameter of the outer strand wires.

TABLE C

| | B | | D | | F | | H | I |
|---|---|---|---|---|---|---|---|---|
| | Cable | C | Ultimate | | Cable | G | Ultimate | % |
| A | diam, | Diam. | tensile | E | diam, | Diam. | tensile | increase |
| Construc- | D | ratio | strength | Construc- | D | ratio | strength | in |
| tion | [mm] | (D/d) | [N] | tion | [mm] | (D/d) | [N] | strength |
| 7 × 7 × 7 | 0.69 | 27 | 455 | 13 × 19-7 × | 0.69 | 27 | 554 | 21.6 |
| | | | | 19-1 × 37 | | | | |
| 7 × 7 × 19 | 1.14 | 45 | 1236 | 16 × 37-16 × | 1.14 | 45 | 1533 | 24.1 |
| | | | | 19-7 × 37 | | | | |
| 7 × (7 × | 1.30 | 51 | 1580 | 19 × 37-20 × | 1.30 | 51 | 1991 | 26.1 |
| 19-1 × 37) | | | | 19-13 × | | | | |
| | | | | 19-7 × | | | | |
| | | | | 19-1 × 37 | | | | |
| 7 × 7 × 37 | 1.60 | 63 | 2407 | 24 × 37-18 × | 1.60 | 63 | 2996 | 24.5 |
| | | | | 37-12 × | | | | |
| | | | | 37-7 × 37 | | | | |

Referring to the first row of Table C, columns A, B, C, D correspond to the 7×7×7 construction of FIG. 6, which has six outer strands 604 and three hundred and forty-three (343) wires, and columns E, F, G, H correspond to the 1.3×19−7×19-1×37 construction of FIG. 14, which has thirteen outer strands 1404 and four hundred and seventeen (417) wires. The construction providing the larger number of outer strands 1404 results in a larger number of wires, which means that a greater volume of metal is contained within the wire rope 1400 having the larger number of outer strands. Table C also shows that the larger number of outer strands results in 21.6 percent greater tensile strength. The wire packing factor for the thirteen outer strand wire rope 1400 of FIG. 114 is 0.572. The wire packing factor for the 7×7×7 construction of FIG. 6 is 0.471. The ratio of overall wire rope diameter to the number of outer strand wire-diameters to span the overall wire rope diameter for the thirteen outer strand wire rope 1400 of FIG. 14 is 27. The ratio of overall wire rope diameter to the number of outer strand wire-diameters to span the overall wire rope diameter for the 7×7×7 construction of FIG. 6 is 27.

Referring to the second row of Table C, columns A, B, C, D correspond to the 7×7×19 construction of FIG. 7, which has six outer strands 704 and nine hundred and thirty-one (931) wires, and columns E, F, G, H correspond to the 16×37-16×19-7×37 construction of FIG. 15, which has sixteen outer strands 1504 and one thousand one hundred and fifty-two (1,152) wires. The construction providing the larger number of outer strands 1504 results in a larger number of wires and a greater volume of metal within the wire rope 1500 having the larger number of outer strands. Table C also shows that the larger number of outer strands results in 24.1 percent greater tensile strength. The wire packing factor for the sixteen outer strand wire rope 1500 of FIG. 15 is 0.570. The wire packing factor for the 7×7×19 construction of FIG. 7 is 0.460. The ratio of overall wire rope diameter to the number of outer strand wire-diameters to span the overall wire rope diameter for the sixteen outer strand wire rope 1500 of FIG. 15 is 45. The ratio of overall wire rope diameter to the number of outer strand wire-diameters to span the overall wire rope diameter for the 7×7×19 construction of FIG. 7 is 45.

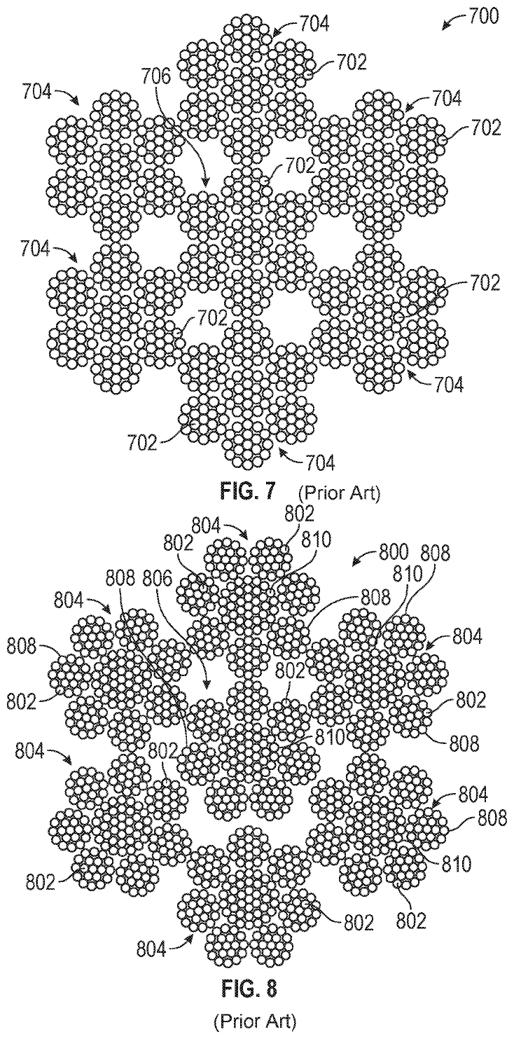

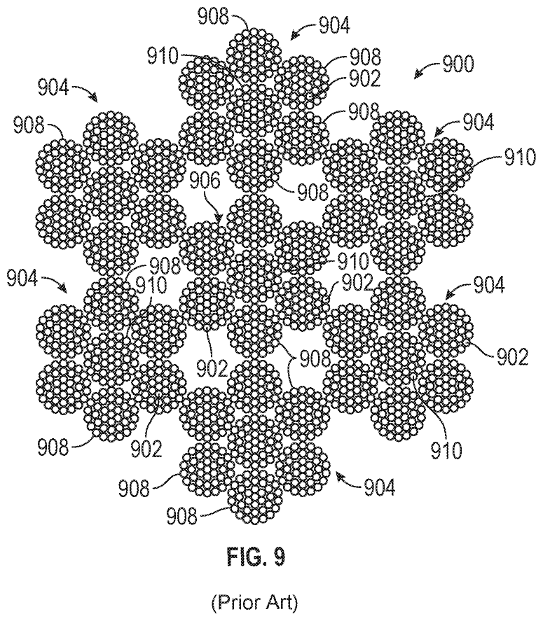

Referring to the third row of Table C, columns A, B, C, D correspond to the 7×(7×19-1×37) construction of FIG. 8, which has six outer strands 704 and one thousand one hundred and ninety (1,190) wires, and columns E, F, G, H correspond to the 19×37-20×19-13×19-7×19-1×37 construction of FIG. 16, which has sixteen outer strands 1604 and one thousand three hundred and sixty-seven (1,367) wires. The construction providing the larger number of outer strands 1604 results in a larger number of wires and a greater volume of metal within the wire rope 1600 having the larger number of outer strands. Table C also shows that the larger number of outer strands results in 26.1 percent greater tensile strength. The wire packing factor for the nineteen outer strand wire rope 1600 of FIG. 16 is 0.577. The wire packing factor for the 7×(7×19-1×37) construction of FIG. 8 is 0.458. The ratio of overall wire rope diameter to the number of outer strand wire-diameters to span the overall wire rope diameter for nineteen outer strand wire rope 1600 of FIG. 16 is 51. The ratio of overall wire rope diameter to the number of outer strand wire-diameters to span the overall wire rope diameter for the 7×7×37 construction of FIG. 8 is 51. Referring to the fourth row of Table C, columns A, B, C, D correspond to the 7×7×37 construction of FIG. 9, which has six outer strands 704 and one thousand eight hundred and thirteen (1,813) wires, and columns E, F, G, H correspond to the 24×37-18×37-12×37-7×37 construction of FIG. 17, which has sixteen outer strands 1704 and two-thousand, two hundred and fifty-seven (2,257) wires. The construction providing the larger number of outer strands 1704 results in a larger number of wires and a greater volume of metal within the wire rope 1700 having the larger number of outer strands. Table C also shows that the larger number of outer strands results in 24.5 percent greater tensile strength. The wire packing factor for the twenty-four outer strand wire rope 1700 of FIG. 17 is 0.569. The wire packing factor for the 7×7×37 construction of FIG. 9 is 0.457. The ratio of overall wire rope diameter to the number of outer strand wire-diameters to span the overall wire rope diameter for nineteen outer strand wire rope 1600 of FIG. 17 is 63 The ratio of overall wire rope diameter to the number of outer strand wire-diameters to span the overall wire rope diameter for the 7×7×37 construction of FIG. 9 is 63.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the disclosure should be limited only by the following claims, and it is appropriate that the claims be construed broadly and in a manner consistent with the scope of the embodiments disclosed herein. The above description is presented to enable any person skilled in the art to create and use a wire rope with enhanced wire wrap. Various modifications to the embodiments will be readily apparent to those skilled in the art, and the generic principles defined herein may be applied to other embodiments and applications without departing from the spirit and scope of the invention. In the preceding description, numerous details are set forth for the purpose of explanation. However, one of ordinary skill in the art will realize that the invention might be practiced without the use of these specific details. In other instances, well-known processes are shown in block diagram form in order not to obscure the description of the invention with unnecessary detail. Identical reference numerals may be used to represent different views of the same or similar item in different drawings.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. A statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about” as used herein can allow for a degree of variability in a value or range, for example, within 10%, within 5%, or within 1% of a stated value or of a stated limit of a range.

Thus, the foregoing description and drawings of embodiments in accordance with the present invention are merely illustrative of the principles of the invention. Therefore, it will be understood that various modifications can be made to the embodiments by those skilled in the art without departing from the spirit and scope of the invention, which is defined in the appended claims.

1. A wire rope comprising:
a core comprising a plurality of core wires; and
thirteen or more outer strands surrounding the core, each outer strand comprising a plurality of outer strand wires, each outer strand wire having a diameter;
wherein the wire rope has a diameter; and
wherein at least twenty-seven outer strand wire diameters are required to span the wire rope diameter.

2. The wire rope of claim 1, wherein:
each core wire has a diameter; and
the core the core wire diameter equals the outer strand wire diameter.

3. The wire rope of claim 1, wherein:
each core wire has a diameter; and
each core wire diameter is in a range of 1.0 to 1.12 times as large as the outer strand wire diameter.

4. The wire rope of claim 1, wherein:
the wire rope has a total wire packing factor of at least 0.54.

5. The wire rope of claim 1, wherein:
the wire rope has a total wire packing factor of at least 0.54; and
the wire rope has a diameter in a range of 0.394 to 2.155 mm.

6. The wire rope of claim 1, wherein:
the wire rope has a total wire packing factor of at least 0.54; and
a range of 27 to 81 outer strand wire diameters are required to span the wire rope diameter.

7. The wire rope of claim 1, wherein:
the wire rope has a total wire packing factor of at least 0.54; and
each outer strand wire diameter is in a range of 0.015 to 0.025 mm.

8. The wire rope of claim 1, wherein:
a range of 27 to 81 outer strand wire diameters are required to span the wire rope diameter.

9. The wire rope of claim 1, wherein:
a range of 27 to 81 outer strand wire diameters are required to span the wire rope diameter; and
the wire rope has a diameter in a range of 0.394 to 2.155 mm.

10. The wire rope of claim 1, wherein:
a range of 27-81 are required to span the wire rope diameter; and
the wires of the outer strands have a diameter in a range of 0.015-0.025 mm.

11. The wire rope of claim 1, wherein:
a range of 27-81 are required to span the wire rope diameter;
the outer strand wires have a diameter in a range of 0.015-0.025 mm; and
wherein a diameter of the core wires is in a range of 1.0 times as large to 1.12 times as large as a diameter of the outer strand wires.

12. The wire rope of claim 1,
wherein the wire rope has a diameter in a range of 0.394-2.155 mm.

13. The wire rope of claim 1,
wherein the wire rope has a diameter in a range of 0.394-2.155 mm; and
wherein the outer strand wires have a diameter in a range of 0.015-0.025 mm.

14. The wire rope of claim 1,
wherein the wire rope has a diameter in a range of 0.394-2.155 mm; and
wherein a diameter of the core is in a range of 0.241-1.697 mm.

15. The wire rope of claim 1,
wherein the wire rope has a total wire packing factor of at least 0.54;
wherein a range of 27-81 are required to span the wire rope diameter;
wherein the wire rope has a diameter in a range of 0.394-2.155 mm; and
wherein a diameter of the core is in a range of 0.241-1.697 mm.

16. The wire rope of claim 1,
wherein the wire rope has a total wire packing factor of at least 0.54;
wherein a range of 27-81 are required to span the wire rope diameter;
wherein the wire rope has a diameter in a range of 0.394-2.155 mm;
wherein a diameter of the core is in a range of 0.241-1.697 mm; and
wherein the outer strand wires have a diameter in a range of 0.015-0.025 mm.

17. The wire rope of claim 1,
wherein the wire rope has a total wire packing factor of at least 0.54;
wherein a range of 27-81 are required to span the wire rope diameter;
wherein the wire rope has a diameter in a range of 0.394-2.155 mm;
wherein a diameter of the core is in a range of 0.241-1.697 mm;
wherein the outer strand wires of the wire rope have a diameter in a range of 0.015-0.025 mm; and
wherein a diameter of each wire of the plurality of wires of the core is in a range of 1.0 times as large to 1.12 times as large as a diameter of each wire of the plurality of wires of the outer strands.

18. The wire rope of claim 1,
wherein the number of outer strands is thirteen; and
wherein the number of outer strand wire-diameters to span the wire rope diameter is twenty-seven.

19. (canceled)

20. (canceled)

21. The wire rope of claim 1,
wherein the number of outer strands is sixteen; and
wherein the number of outer strand wire-diameters to span the rope diameter is 45.

22. (canceled)

23. (canceled)

24. The wire rope of claim 1,
wherein the number of outer strands is nineteen;
wherein the number of outer strand wire-diameters to span the wire rope diameter is 51.

25. (canceled)

26. (canceled)

27. The wire rope of claim 1,
wherein the number of outer strands is twenty-four outer strands that each includes a plurality of wires;
wherein the number of outer strand wire-diameters to span the wire rope diameter is 63.

28. (canceled)

29. (canceled)

30. A surgical instrument comprising:
a wire rope, a shaft comprising a first end and a second end, an end effector coupled to the first end of the shaft, and an actuator coupled to the second end of the shaft;
wherein the wire rope comprises an inner core and an outer wrap layer surrounding the inner core;
wherein the inner core comprises a plurality of core wires of a first material, and the inner core has a diameter in a range of 0.241 to 1.697 mm;
wherein the outer wrap layer comprises thirteen or more outer strands, each of the thirteen or more outer strands comprising a plurality of outer strand wires, each of the outer strand wires comprising the first material, and each of the outer strand wires having a diameter in a range of 0.046 to 0.229 mm; wherein the shaft has a diameter in a range of 4 to 10 mm;
wherein the end effector comprises a bend portion and a guide, the guide is positioned to guide the wire rope about the bend portion through an angle of at least 15 degrees, and a largest diameter of the end effector is less than or equal to the diameter of the shaft; and
wherein the wire rope is coupled to the actuator, and a tensile force upon the wire rope from the actuator is in a range of 44 to 445 N with a strain smaller than 0.02.

31-67. (canceled)
